(19) United States
(12) Patent Application Publication
(10) Pub. No.: US 2024/0189213 A1
(43) Pub. Date: Jun. 13, 2024

(54) AGENTS FOR DYEING KERATINOUS MATERIAL, IN PARTICULAR HUMAN HAIR, CONTAINING AMINOSILICONES, PIGMENTS AND PARTICULAR ALKYLENE GLYCOLS

(71) Applicant: HENKEL AG & CO. KGAA, Düsseldorf (DE)

(72) Inventors: Constanze KRUCK, Grevenbroich (DE); Sandra HILBIG, Bochum (DE); Melanie MOCH, Dormagen (DE); Gabriele WESER, Essen (DE); Ulrike SCHUMACHER, Duesseldorf (DE); Imme BREUER, Duesseldorf (DE)

(21) Appl. No.: 18/284,886

(22) PCT Filed: Mar. 25, 2022
(86) PCT No.: PCT/EP2022/057872

§ 371 Date: Sep. 29, 2023

(30) Foreign Application Priority Data
Apr. 20, 2021 (DE) 10 2021 203 918.9

(51) Int. Cl. A61K 8/898; A61K 8/49; A61K 8/60; A61K 8/86; A61K 8/9794; A61Q 5/06

(52) U.S. Cl. CPC A61K 8/898; A61K 8/4913; A61K 8/4926; A61K 8/602; A61K 8/604; A61K 8/86; A61K 8/9794; A61Q 5/065; A61K 2800/43

(57) ABSTRACT

An agent for dyeing keratinous material, such as human hair, may include: at least alkylene glycol of formula (AG)

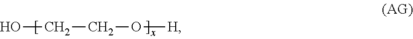

where x is an integer ranging from 1 to 10,000.

The agent may further include at least one pigment and at least one amino-functionalized silicone polymer.

### CROSS-REFERENCE TO RELATED APPLICATIONS

The present application is a national stage entry according to 35 U.S.C. § 371 of PCT application No.: PCT/EP2022/057872 filed on Mar. 25, 2022; which claims priority to German patent application 10 2021 203 918.9 filed on Apr. 20, 2021; all of which are incorporated herein by reference in their entirety and for all purposes.

### TECHNICAL FIELD

The subject matter of the present application is an agent for dyeing keratinous material, in particular human hair, which contains at least one alkylene glycol of formula (AG) (a1), at least one pigment (a2), and at least one amino-functionalized silicone polymer (a3).

A further subject matter of this application is a method for dyeing keratinous material, in particular human hair, wherein the agent described above is applied to the keratinous material and, if appropriate, is rinsed out again after an exposure time of 30 seconds to 45 minutes.

### BACKGROUND

Changing the shape and color of keratinous material, in particular human hair, represents an important field of modern cosmetics. The person skilled in the art knows a variety of coloring systems for changing hair color, depending on the coloring requirements. Oxidation dyes are typically used for permanent, intense dyeing with good fastness properties and good gray coverage. Such coloring agents contain oxidation dye precursors, what are known as developer components and coupler components, which together form the actual dyes under the influence of oxidizing agents such as, for example, hydrogen peroxide. Oxidation dyes are characterized by very long-lasting color results.

When using direct dyes, already-formed dyes diffuse from the coloring agent into the hair fiber. In comparison with oxidative hair coloring, the colors obtained with direct dyes have a lower durability and a more rapid washing out. Colorings with direct dyes usually remain on the hair for a period of between 5 and 20 hair washes.

The use of color pigments for brief color changes on the hair and/or the skin is known. Color pigments are generally understood to mean insoluble coloring substances. These are present undissolved in the form of small particles in the coloring formulation, and are merely deposited externally on the hair fibers and/or skin surface. Therefore, they can normally be removed again without residue by a few washes with surfactant-containing cleaning agents. Various products of this type are available on the market under the name of hair mascara.

If the user desires particularly long-lasting colorings, the use of oxidative coloring agents has hitherto been the only option. However, despite multiple optimization attempts, an unpleasant ammonia odor or amine odor cannot be completely avoided in oxidative hair dyeing. The hair damage that remains associated with the use of the oxidative coloring agents also has a disadvantageous effect on the hair of the user. A continuing challenge is therefore the search for alternative, high-performance dyes and dyeing processes. Recently, there has been a particular focus on dyeing systems based on pigments.

### SUMMARY

It was the object of the present invention to provide a coloring agent which makes it possible to fix pigments to the hair in an extremely durable manner. When using the agent in a dyeing method, particularly intensive dyeing results with good wash fastness, good rubbing fastness, good smoothing capability, and a particularly uniform color result should be achieved.

Surprisingly, it has been found that the aforementioned object can be achieved in an outstanding manner when keratinous material, in particular human hair, is colored with an agent which contains at least alkylene glycol of formula (AG) (a1), at least one pigment (a2), and at least one amino-functionalized silicone polymer (a3).

A first subject matter of the present invention is an agent for dyeing keratinous material, in particular human hair, containing:
(a1) at least alkylene glycol of formula (AG)

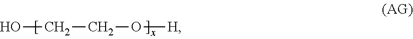

where
X is an integer from 1 to 10,000,
(a2) at least one pigment, and
(a3) at least one amino-functionalized silicone polymer.

Within the context of the work leading to the present invention, it was found that especially good color results were obtained when the pigment or pigments, in admixture with at least one alkylene glycol of the formula (AG) and at least one amino-silicone, were applied to the keratinous material, in particular human hair. In particular, the wash fastness and the rubbing fastness of the colored hair could be improved.

### DETAILED DESCRIPTION

### Keratinous Material

Keratinous material is understood to mean hair, skin, and nails (such as, for example, fingernails and/or toenails). Furthermore, wool, furs, and feathers also fall under the definition of keratinous material.

Keratinous material is preferably understood to be human hair, human skin, and human nails, in particular fingernails and toenails. Keratinous material is very particularly preferably understood to mean human hair.

### Coloring Agent

Within the scope of this invention, the term “coloring agent” is used for a coloring of the keratinous material, in particular hair, brought about by use of pigments. With this coloring, the pigments as coloring compounds are deposited in a particularly homogeneous and uniform film on the surface of the keratinous material.

According to the invention, the coloring agent represents a ready-to-use agent. This ready-to-use agent can, for example, be filled into a container and applied to the keratinous material in this form without further dilution, mixing, or other method steps. For reasons of storage stability, however, it has been found to be very particularly preferred if the ready-to-use cosmetic agent is produced by the hairdresser or user only shortly before use. To prepare the ready-to-use agent, for example, the mixture or the pre-dispersion of alkylene glycol (AG) (a1) and pigment (a2) with one or more additional agents can take place, wherein one of these additional agents contains at least one amino-functionalized silicone polymer (a3). However, it is also just as conceivable that the ready-to-use agent is produced by mixing at least three different agents, wherein one of these agents contains at least one alkylene glycol (AG) (a1), an additional agent contains at least one pigment (a2), and still another agent contains at least amino-functionalized silicone polymer (a3). The means can be mixed by being shaken, for example, and in this way ensures a very particularly uniform distribution of the dispersed pigments.

In other words, a first subject matter of the present invention is an agent for dyeing keratinous material, in particular human hair, containing, in a cosmetic carrier:
(a1) at least alkylene glycol of formula (AG)

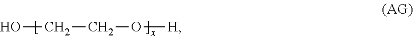

where
x is an integer from 1 to 10,000, and
(a2) is at least one pigment, and
(a3) at least one amino-functionalized silicone polymer.

### Alkylene Glycols of Formula (AG-I) (a1)

As the second component essential to the invention, the agent according to the invention contains at least one alkylene glycol of formula (I),

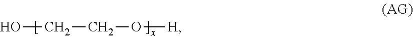

where
x is an integer of from 1 to 10,000.

Surprisingly, it has been found that the use of at least one specific alkylene glycol of formula (AG) greatly improves the washing fastness and the rub fastness of the colorings after applying the agent according to the invention on the keratinous material.

The alkylene glycols of the formula (AG) are protic substances having at least one hydroxy group which, due to their repeating-CH2-CH2-O— unit, can also be referred to as polyethylene glycols insofar as x is a value of at least 2. In the alkylene glycols (a1) of formula (AG), x is an integer from 1 to 10,000. In the context of the work leading to this invention, it was found that these polyethylene glycols exhibit particularly favorable suitability for, on the one hand, improving the fastness properties of the coloring agents and, on the other hand, also optimally adjusting the viscosity of the agents.

Depending on their chain length, polyethylene glycols are liquid or solid water-soluble polymers. Polyethylene glycols with a molecular mass between 200 g/mol and 400 g/mol are non-volatile liquids at room temperature. PEG 600 has a melting range of from 17 to 22° C., and therefore a pasty consistency. With molecular masses above 3000 g/mol, the PEGs are solid substances and are commercially available as flakes or powders.

Especially the use of low molecular weight alkylene glycols (or, respectively, polyethylene glycols) has proven to be well-suited for achieving the object according to the invention. In the event of low molecular weight alkylene glycols (or, respectively, polyethylene glycols) in the context of the present invention, x1 denotes an integer from 1 to 100, preferably an integer from 1 to 80, more preferably an integer from 2 to 60, even more preferably an integer from 3 to 40, even more preferably an integer from 4 to 20, and very particularly preferably an integer from 6 to 15.

Within the scope of another very particularly preferred embodiment, an agent according to the invention is characterized in that it contains:
(a1) at least one alkylene glycol of formula (AG-1), where

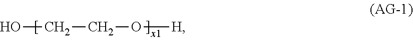

x1 is an integer from 1 to 100, preferably an integer from 1 to 80, more preferably an integer from 2 to 60, even more preferably an integer from 3 to 40, even more preferably an integer from 4 to 20, and very particularly preferably an integer from 6 to 15.

A very particularly preferred low molecular weight polyethylene glycol is PEG-8, for example. PEG-8 comprises, on average, 8 ethylene glycol units (x1=8), has an average molecular weight of 400 g/mol, and bears the CAS number 25322-68-3. PEG-8 is alternatively also referred to as PEG 400 and is commercially available, for example from APS.

Additional well-suited low molecular weight polyethylene glycols are, for example, PEG-6, PEG-7, PEG-9 and PEG-10.

Another well-suited polyethylene glycol is PEG-32, for example. PEG-32 comprises 32 ethylene glycol units (x1=32), has an average molar mass of 1500 g/mol and bears the CAS number 25322-68-3. PEG-32 is alternatively also referred to as PEG 1500 and can, for example, be purchased commercially from Clariant.

Furthermore, the use of high molecular weight polyethylene glycols for achieving the object according to the invention has also proven to be well suited.

High molecular weight polyethylene glycols within the meaning of the present invention can be represented by the formula (AG-2), wherein the index number x2 stands for an integer from 101 to 10,000

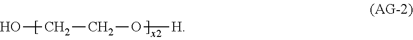

In the case of very well-suited high molecular weight polyethylene glycols, x2 denotes an integer from 101 to 1000, preferably an integer from 105 to 800, more preferably an integer from 107 to 600, even more preferably an integer from 109 to 400, and very particularly preferably an integer from 110 to 200.

Within the scope of another very particularly preferred embodiment, an agent according to the invention is characterized in that it contains:
(a1) at least one alkylene glycol of formula (AG-2), where

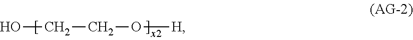

x2 denotes an integer from 101 to 1000, preferably an integer from 105 to 800, more preferably an integer from 107 to 600, even more preferably an integer from 109 to 400, and very particularly preferably an integer from 110 to 200.

A very particularly well-suited high molecular weight polyethylene glycol is, for example, PEG 6000, which can be obtained commercially from the National Starch company (China). The molecular weight of PEG 6000 is 6000 to 7500 g/mol, corresponding to an x2 value of 136 to 171.

Another well-suited polyethylene glycol is PEG 12000, which, for example, is commercially sold by CG chemicals under the trade name of Polyethylene Glycol 12000 S (or PEG 12000 S). The molecular weight of PEG 12000 is given at 10,500 to 15,000 g/mol, corresponding to an x2 value of 238 to 341.

Another well-suited polyethylene glycol is also PEG 20000. which is commercially available under the trade name Polyglycol 20000 P or under the alternative name PEG-350 from Clariant. For PEG 20000, an average molecular weight of 20,000 g/mol is given, which corresponds to an x2 value of 454.

Surprisingly, it has been found that coloring agents which contain both a low molecular weight polyethylene glycol and a high molecular weight polyethylene glycol have particularly favorable application properties, since these agents have both very good fastness properties and are optimized with regard to their rheological profile.

Within the scope of another very particularly preferred embodiment, an agent according to the invention is characterized in that it contains:
(a11) at least one first alkylene glycol of formula (AG-1), where

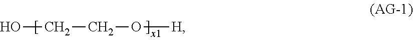

x1 denotes an integer from 1 to 100, preferably an integer from 1 to 80, more preferably an integer from 2 to 60, even more preferably an integer from 3 to 40, even more preferably an integer from 4 to 20, and very particularly preferably an integer from 6 to 15, and
(a12) at least one second alkylene glycol of formula (AG-2), where

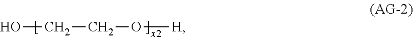

x2 denotes an integer from 101 to 1000, preferably an integer from 105 to 800, more preferably an integer from 107 to 600, even more preferably an integer from 109 to 400, and very particularly preferably an integer from 110 to 200.

To further optimize the application properties, the agent according to the invention comprises the alkylene glycol(s) (AG), preferably in certain quantity ranges which, for example—based on the total weight of the agent—can be in the range of from 10.0 to 99.0% by weight, preferably 30.0 to 99.0% by weight, more preferably 50.0 to 99.0% by weight, and very particularly preferably 70.0 to 99.0% by weight.

Within the scope of another very particularly preferred embodiment, an agent according to the invention is characterized in that it contains—based on the total weight of the agent—one or more alkylene glycols corresponding to formula (AG) (a1) in a total amount of 10.0 to 99.0 wt. %, preferably 30.0 to 99.0 wt. %, more preferably 50.0 to 99.0 wt. % and very particularly preferably 70.0 to 99.0 wt. %.

The agent according to the invention preferably contains—based on the total weight of the agent—(a11) one or more alkylene glycols of formula (AG-1) in a total amount of 20.0 to 99.0% by weight, preferably 40.0 to 95.0% by weight, particularly preferably 60.0 to 90.0% by weight.

The agent according to the invention preferably contains—based on the total weight of the agent—(a12) one or more alkylene glycols of formula (AG-2) in a total amount of 1.0 to 35.0% by weight, preferably 3.0 to 30.0% by weight, particularly preferably 4.0 to 25.0% by weight.

Within the scope of another very particularly preferred embodiment, an agent according to the invention is characterized in that it contains—based on the total weight of the agent—(a1) one or more alkylene glycols of formula (AG-1) in a total amount of 20.0 to 99.0% by weight, preferably 40.0 to 95.0% by weight, more preferably 60.0 to 90.0% by weight, and/or contains one or more alkylene glycols of formula (AG-2) in a total amount of 1.0 to 35.0% by weight, preferably 3.0 to 30.0% by weight, and particularly preferably 4.0 to 25.0% by weight.

Within the scope of another very particularly preferred embodiment, an agent according to the invention is characterized in that it contains—based on the total weight of the agent—(a11) one or more alkylene glycols of formula (AG-1) in a total amount of 20.0 to 99.0% by weight, and
(a12) one or more alkylene glycols of formula (AG-2) in a total amount of 1.0 to 35.0% by weight.

Within the scope of another very particularly preferred embodiment, an agent according to the invention is characterized in that it contains—based on the total weight of the agent—(a11) one or more alkylene glycols of formula (AG-1) in a total amount of 40.0 to 95.0% by weight, and
(a12) one or more alkylene glycols of formula (AG-2) in a total amount of 3.0 to 30.0% by weight.

Within the scope of another very particularly preferred embodiment, an agent according to the invention is characterized in that it contains—based on the total weight of the agent—(a11) one or more alkylene glycols of formula (AG-1) in a total amount of 60.0 to 99.0% by weight, and
(a12) one or more alkylene glycols of formula (AG-2) in a total amount of 4.0 to 25.0% by weight.

It is hereby understood that the sum of all of the alkylene glycols (AG) (a1), pigments (a2), and amino-functionalized silicone polymers (a3) contained in the agents cannot be any more than 100% by weight. If still further, optional ingredients are to be used in the agent, the total sum of (a1), (a2), and (a3) decreases to a corresponding extent to values of less than 100% by weight.

### Pigments (a2)

As the second essential component, the agent according to the invention contains at least one pigment (a2). Pigments within the meaning of the present invention are understood to mean dyeing compounds which have a solubility of less than 0.5 g/L, preferably of less than 0.1 g/L, even more preferably of less than 0.05 g/L, at 25° C. in water. The method described below, for example, can be used to determine water solubility: 0.5 g of the pigment is weighed out in a beaker. A stir bar is added. Then one liter of distilled water is added. This mixture is heated to 25° C. while stirring with a magnetic stirrer for one hour. If still undissolved components of the pigment are visible in the mixture after this period, the solubility of the pigment is below 0.5 g/L. If the pigment-water mixture cannot be visually assessed due to the high intensity of the pigment that may be finely dispersed, the mixture is filtered. If a portion of undissolved pigments remains on the filter paper, the solubility of the pigment is below 0.5 g/L.

Suitable color pigments can be of inorganic and/or organic origin.

In a preferred embodiment, an agent according to the invention is characterized in that it contains at least one dyeing compound (a2) from the group consisting of inorganic and/or organic pigments.

Preferred color pigments are selected from synthetic or natural inorganic pigments. Inorganic color pigments of natural origin can be produced, for example, from chalk, ocher, umbra, green soil, burnt sienna, or graphite. Furthermore, black pigments such as, for example, iron oxide black; chromatic pigments such as, for example, ultramarine or iron oxide red; and also fluorescent or phosphorescent pigments can be used as inorganic color pigments.

Colored metal oxides, hydroxides and oxide hydrates, mixed phase pigments, sulfur-containing silicates, silicates, metal sulfides, complex metal cyanides, metal sulfates, chromates, and/or molybdates are particularly suitable. Particularly preferred color pigments are black iron oxide (CI 77499), yellow iron oxide (CI 77492), red and brown iron oxide (CI 77491), manganese violet (CI 77742), ultramarine (sodium aluminum sulfosilicates, CI 77007, Pigment Blue 29), chromium oxide hydrate (CI 77289), iron blue (ferric ferrocyanide, CI 77510), and/or carmine (cochineal).

Color pigments which are likewise particularly preferred according to the invention are colored pearlescent pigments. These are usually based on mica and may be coated with one or more metal oxides. Mica is a phyllosilicate. The most important representatives of these silicates are muscovite, phlogopite, paragonite, biotite, lepidolite, and margarite. In order to produce the pearlescing pigments in conjunction with metal oxides, mica, primarily muscovite or phlogopite, is coated with a metal oxide.

As an alternative to natural mica, synthetic mica coated with one or more metal oxides(s) can also be used as a pearlescent pigment. Particularly preferred pearlescent pigments are based on natural or synthetic mica and are coated with one or more of the aforementioned metal oxides. The color of the respective pigments can be varied by varying the layer thickness of the metal oxide(s).

In another preferred embodiment, an agent according to the invention is characterized in that it contains at least one inorganic pigment (a2) preferably selected from the group consisting of colored metal oxides, metal hydroxides, metal oxide hydrates, silicates, metal sulfides, complex metal cyanides, metal sulfates, bronze pigments, and/or from mica-based colored pigments which are coated with at least one metal oxide and/or a metal oxychloride.

In another preferred embodiment, an agent according to the invention is characterized in that it contains at least one dyeing compound (a2) from the group consisting of pigments selected from mica-based colored pigments which are coated with one or more metal oxides from the group consisting of titanium dioxide (CI 77891), black iron oxide (CI 77499), yellow iron oxide (CI 77492), red and/or brown iron oxide (CI 77491, CI 77499), manganese violet (CI 77742), ultramarine (sodium aluminum sulfosilicates, CI 77007, pigment blue 29), chromium oxide hydrate (CI 77289), chromium oxide (CI 77288), and/or iron blue (ferric ferrocyanide, CI 77510).

Examples of particularly suitable color pigments are commercially available, for example, under the trade names Rona®, Colorona®, Xirona®, Dichrona®, and Timiron® from the company Merck; Ariabel® and Unipure® from the company Sensient; Prestige® from the company Eckart Cosmetic Colors; and Sunshine® from the company Sunstar.

Very particularly preferred color pigments with the trade name Colorona® are, for example:
Colorona Copper, Merck, MICA, CI 77491 (IRON OXIDES)
Colorona Passion Orange, Merck, Mica, CI 77491 (Iron Oxides), Alumina
Colorona Patina Silver, Merck, MICA, CI 77499 (IRON OXIDES), CI 77891 (TITANIUM DIOXIDE)
Colorona RY, Merck, CI 77891 (TITANIUM DIOXIDE), MICA, CI 75470 (CARMINE)
Colorona Oriental Beige, Merck, MICA, CI 77891 (TITANIUM DIOXIDE), CI 77491 (IRON OXIDES)
Colorona Dark Blue, Merck, MICA, TITANIUM DIOXIDE, FERRIC FERROCYANIDE
Colorona Chameleon, Merck, CI 77491 (IRON OXIDES), MICA
Colorona Aborigine Amber, Merck, MICA, CI 77499 (IRON OXIDES), CI 77891 (TITANIUM DIOXIDE)
Colorona Blackstar Blue, Merck, CI 77499 (IRON OXIDES), MICA
Colorona Patagonian Purple, Merck, MICA, CI 77491 (IRON OXIDES), CI 77891 (TITANIUM DIOXIDE), CI 77510 (FERRIC FERROCYANIDE)
Colorona Red Brown, Merck, MICA, CI 77491 (IRON OXIDES), CI 77891 (TITANIUM DIOXIDE)
Colorona Russet, Merck, CI 77491 (TITANIUM DIOXIDE), MICA, CI 77891 (IRON OXIDES)
Colorona Imperial Red, Merck, MICA, TITANIUM DIOXIDE (CI 77891), D&C RED NO. 30 (CI 73360)
Colorona Majestic Green, Merck, CI 77891 (TITANIUM DIOXIDE), MICA, CI 77288 (CHROMIUM OXIDE GREENS)
Colorona Light Blue, Merck, MICA, TITANIUM DIOXIDE (CI 77891), FERRIC FERROCYANIDE (CI 77510)
Colorona Red Gold, Merck, MICA, CI 77891 (TITANIUM DIOXIDE), CI 77491 (IRON OXIDES)
Colorona Gold Plus MP 25, Merck, MICA, TITANIUM DIOXIDE (CI 77891), IRON OXIDES (CI 77491)
Colorona Carmine Red, Merck, MICA, TITANIUM DIOXIDE, CARMINE
Colorona Blackstar Green, Merck, MICA, CI 77499 (IRON OXIDES)
Colorona Bordeaux, Merck, MICA, CI 77491 (IRON OXIDES)
Colorona Bronze, Merck, MICA, CI 77491 (IRON OXIDES)
Colorona Bronze Fine, Merck, MICA, CI 77491 (IRON OXIDES)
Colorona Fine Gold MP 20, Merck, MICA, CI 77891 (TITANIUM DIOXIDE), CI 77491 (IRON OXIDES)
Colorona Sienna Fine, Merck, CI 77491 (IRON OXIDES), MICA
Colorona Sienna, Merck, MICA, CI 77491 (IRON OXIDES)
Colorona Precious Gold, Merck, Mica, CI 77891 (Titanium dioxide), Silica, CI 77491 (IRON OXIDES), Tin oxide
Colorona Sun Gold Sparkle MP 29, Merck, MICA, TITANIUM DIOXIDE, IRON OXIDES, MICA, CI 77891, CI 77491 (EU)
Colorona Mica Black, Merck, CI 77499 (Iron oxides), Mica, CI 77891 (Titanium dioxide)
Colorona Bright Gold, Merck, Mica, CI 77891 (Titanium dioxide), CI 77491(Iron oxides)
Colorona Blackstar Gold, Merck, MICA, CI 77499 (IRON OXIDES)

Additional particularly preferred color pigments with the trade name Xirona® are, for example:
Xirona Golden Sky, Merck, Silica, CI 77891 (Titanium Dioxide), Tin Oxide
Xirona Caribbean Blue, Merck, Mica, CI 77891 (Titanium Dioxide), Silica, Tin Oxide
Xirona Kiwi Rose, Merck, Silica, CI 77891 (Titanium Dioxide), Tin Oxide
Xirona Magic Mauve, Merck, Silica, CI 77891 (Titanium Dioxide), Tin Oxide.

In addition, particularly preferred color pigments with the trade name Unipure® are, for example:
Unipure Red LC 381 EM, Sensient CI 77491 (Iron Oxides), Silica
Unipure Black LC 989 EM, Sensient, CI 77499 (Iron Oxides), Silica
Unipure Yellow LC 182 EM, Sensient, CI 77492 (Iron Oxides), Silica

Within the scope of another embodiment, the agent according to the invention can also contain (a2) one or more dyeing compounds from the group consisting of organic pigments.

The organic pigments according to the invention are correspondingly insoluble organic dyes or color lakes which may be selected, for example, from the group of nitroso, nitro, azo, xanthene, anthraquinone, isoindolinone, isoindoline, quinacridone, perinone, perylene, diketopyrrolopyorrole, indigo, thioindido, dioxazine, and/or triarylmethane compounds.

Particularly well suited organic pigments can, for example, include carmine, quinacridone, phthalocyanine, sorghum, blue pigments with the Color Index numbers CI 42090, CI 69800, CI 69825, CI 73000, CI 74100, or CI 74160, yellow pigments with the Color Index numbers CI 11680, CI 11710, CI 15985, CI 19140, CI 20040, CI 21100, CI 21108, CI 47000, or CI 47005, green pigments with the Color Index numbers CI 61565, CI 61570, or CI 74260, orange pigments with the Color Index numbers CI 11725, CI 15510, CI 45370, or CI 71105, and red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915, and/or CI 75470.

In another particularly preferred embodiment, an agent according to the invention is characterized in that it contains at least one organic pigment (a2) preferably selected from the group consisting of carmine, quinacridone, phthalocyanine, sorghum, blue pigments with the Color Index numbers CI 42090, CI 69800, CI 69825, CI 73000, CI 74100, or CI 74160, yellow pigments with the Color Index numbers CI 11680, CI 11710, CI 15985, CI 19140, CI 20040, CI 21100, CI 21108, CI 47000, or CI 47005, green pigments with the Color Index numbers CI 61565, CI 61570, or CI 74260, orange pigments with the Color Index numbers CI 11725, CI 15510, CI 45370, or CI 71105, and red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915, and/or CI 75470.

The organic pigment can furthermore also be a color lake. The term color lake within the scope of the invention is understood to mean particles which comprise a layer of absorbed dyes, wherein the unit consisting of particles and dye is insoluble under the aforementioned conditions. The particles may be, for example, inorganic substrates which may be aluminum, silica, calcium borosilicate, calcium aluminum borosilicate, or aluminum.

For example, the alizarin color lake can be used as the color lake.

Owing to their excellent light and temperature resistance, the use of the aforementioned pigments in the method according to the invention is very particularly preferred. It is further preferred if the pigments used have a certain particle size. It is therefore advantageous according to the invention if the at least one pigment has an average particle size D₅₀ from 1.0 to 50 μm, preferably from 5.0 to 45 μm, preferably from 10 to 40 μm, in particular from 14 to 30 μm. The average particle size D₅₀ can be determined, for example, using dynamic light scattering (DLS).

The pigments (a2) represent the second essential component of the agent according to the invention and are preferably used within certain quantity ranges in the agent.

Particularly positive results were obtained when the agent contained—based on the total weight of the agent—one or more pigments (a2) in a total amount of 0.01 to 10.0% by weight, preferably 0.1 to 5.0% by weight, more preferably 0.2 to 2.5% by weight, and very particularly preferably 0.25 to 1.5% by weight.

In another very particularly preferred embodiment, an agent according to the invention is characterized in that the agent contains—based on the total weight of the agent—one or more pigments (a2) in a total amount of 0.01 to 10.0% by weight, preferably 0.1 to 5.0% by weight, more preferably 0.2 to 2.5% by weight, and very particularly preferably 0.25 to 1.5% by weight.

In another very particularly preferred embodiment, an agent according to the invention is characterized in that the agent contains—based on the total weight of the agent—one or more inorganic pigments (a2) in a total amount of 0.01 to 10.0% by weight, preferably 0.1 to 5.0% by weight, more preferably 0.2 to 2.5% by weight, and very particularly preferably 0.25 to 1.5% by weight.

In another very particularly preferred embodiment, an agent according to the invention is characterized in that the agent contains—based on the total weight of the agent—one or more organic pigments (a2) in a total amount of 0.01 to 10.0% by weight, preferably 0.1 to 5.0% by weight, more preferably 0.2 to 2.5% by weight, and very particularly preferably 0.25 to 1.5% by weight.

### Amino-Functionalized Silicone Polymers (a3)

As a third ingredient essential to the invention, the agent according to the invention contains at least one amino-functionalized silicone polymer (a3). The amino-functionalized silicone polymer can alternatively also be referred to as amino silicones or amodimethicone.

Silicone polymers are generally macromolecules with a molecular weight of at least 500 g/mol, preferably at least 1000 g/mol, more preferably at least 2500 g/mol and more preferably at least 5000 g/mol which comprise repeating organic units.

The maximum molecular weight of the silicone polymer depends on the degree of polymerization (number of polymerized monomers), and the batch size, and is also determined by the polymerization method. In the context of the present invention, it is preferable if the maximum molecular weight of the silicone polymers is not more than 10⁷ g/mol, preferably not more than 10⁶ g/mol, and particularly preferably not more than 10⁵ g/mol.

The silicone polymers comprise many Si—O repeat units, wherein the Si atoms can bear organic residues such as, for example, alkyl groups or substituted alkyl groups. Alternatively, a silicone polymer is therefore also referred to as polydimethylsiloxane.

Corresponding to the high molecular weight of the silicone polymers, these are based on more than 10 Si—O repeat units, preferably more than 50 Si—O repeat units, and particularly preferably more than 100 Si—O repeat units, very particularly preferably more than 500 Si—O units.

An amino-functionalized silicone polymer is understood to mean a functionalized silicone which bears at least one structural unit with an amino group. The amino-functionalized silicone polymer preferably bears a plurality of structural units with at least one amino group in each instance. An amino group is understood to mean a primary amino group, a secondary amino group, and a tertiary amino group. All these amino groups can be protonated in an acidic environment and are then present in their cationic form.

In principle, it was possible to achieve positive effects of amino-functionalized silicone polymers (a3) when they bore at least one primary, at least one secondary, and/or at least one tertiary amino group. However, colorings with the highest color intensities were observed when an amino-functionalized silicone polymer (a3) was used in an agent containing at least one secondary amino group.

In a very particularly preferred embodiment, an agent according to the invention is
characterized in that it contains
(a3) at least one amino-functionalized silicone polymer having at least one secondary amino group.

The secondary amino groups(s) can be at different positions in the amino-functionalized silicone polymer. A particularly good effect was found when an amino-functionalized silicone polymer (a3) was used that had at least one, preferably several, structural units of the formula (Si-amino).

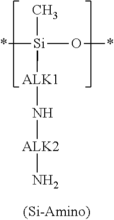

In the structural units of formula (Si-amino), the abbreviations ALK and ALK2 are each independently a linear or branched divalent C₁-C₂₀ alkylene group.

In an additional very particularly preferred embodiment, an agent according to the invention is characterized in that the agent contains at least one amino-functionalized silicone polymer (a3) which comprises at least one structural unit of formula (Si-amino),

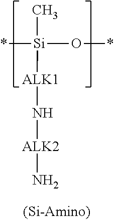

where
ALK1 and ALK2 represent, independently of one another, a linear or branched divalent C₁-C₂₀ alkylene group.

The position marked with an asterisk (*) always indicates the bond to other structural units of the silicone polymer. For example, the silicon atom adjacent to the asterisk can be bonded to an additional oxygen atom, and the oxygen atom adjacent to the asterisk can be bonded to an additional silicon atom or else to a C₁-C₆ alkyl group.

A divalent C₁-C₂₀ alkylene group can alternatively also be termed a double-bond C₁-C₂₀ alkylene group, which means that each moiety ALK1 or ALK2 can have two bonds.

In the case of ALK1, the silicon atom is bonded to the moiety ALK1, and the second bond is between ALK1 and the secondary amino group.

In the case of ALK2, the secondary amino group bonds with the moiety ALK2, and the second bond is formed between ALK2 and the primary amino group.

Examples of a linear divalent C₁C₂₀ alkylene group are, for example, the methylene group (—CH₂), the ethylene group (—CH—CH₂—CH₂), the propylene group (—CH—CH₂—CH₂—CH₂—), and the butylene group (—CH—CH₂—CH₂—CH₂—CH₂—). The propylene group (—CH₂—CH₂—CH₂—) is particularly preferred. Starting at a chain length of 3 C atoms, divalent alkylene groups may also be branched. Examples of branched, divalent C₃C₂₀ alkylene groups are (—CH₂—CH(CH₃)—) and (—CH₂—CH(CH₃)—CH₂—).

In an additional particularly preferred embodiment, the structural units of the formula (Si-amino) represent repeat units in the amino-functionalized silicone polymer (a3), so that the silicone polymer comprises several structural units of formula (Si-amino).

In the following, particularly well-suited amino-functionalized silicone polymers (a3) with at least one secondary amino group are listed.

Colorings with the greatest color intensities were obtainable when an agent was applied on the keratinous material, which agent contains at least one amino-functionalized silicone polymer (a3) which comprises structural units of formula (Si-I) and formula (Si-II):

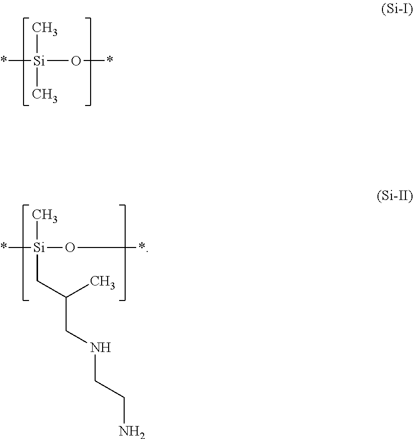

In an additional, explicitly very particularly preferred embodiment, an agent according to the invention is characterized in that it contains at least one amino-functionalized silicone polymer (a3) which comprises structural units of formula (Si-I) and formula (Si-II):

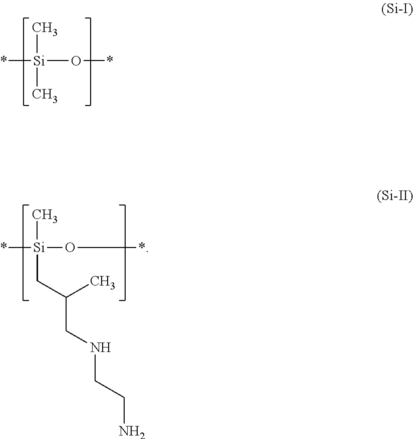

A corresponding amino-functionalized silicone polymer with the structural units (Si-I) and (Si-II) is, for example, the commercial product DC 2-8566 or Dowsil 2-8566 Amino Fluid, which is sold commercially by Dow Chemical Company and which bears the designation “Siloxanes and Silicones, 3-[(2-Aminoethyl)amino]-2-methylpropyl Me, Di-Me-Siloxane” and the CAS number 106842-44-8. Another amino-functionalized silicone polymer with the structural units (Si-I) and (Si-II) is, for example, the commercial product DOWSIL™ AP-8568 Amino Fluid, which is likewise sold commercially by Dow Chemical Company.

Within the scope of another preferred embodiment, an agent according to the invention is characterized in that it contains at least one amino-functional silicone polymer (a3) of the formula of the formula (Si-III):

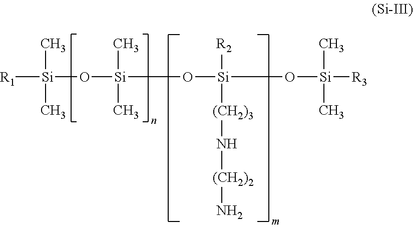

where
m and n denote numbers that are selected such that the sum (n+m) is in a range of from 1 to 1000,
n is a number in a range of from 0 to 999 and m is a number in a range of from 1 to 1000,
R1, R2, and R3, which are identical or different, denote a hydroxyl group or a C1-4 alkoxy group,
at least one of the R1 to R3 groups denoting a hydroxyl group.

Additional agents preferred according to the invention are characterized by their content of at least one amino-functional silicone polymer (a3) of formula (Si-IV),

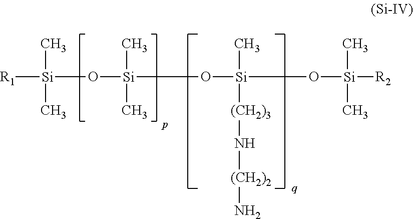

where
p and q denote numbers that are selected such that the sum (p+q) is in a range of from 1 to 1000,
p is a number in a range of from 0 to 999 and q is a number in a range of from 1 to 1000, and
R1 and R2, which are different, denote a hydroxy group or a C1-4 alkoxy group, at least one of the groups R1 to R2 denoting a hydroxy group.

The silicones of formula (Si-III) and (Si-IV) are different due to the moiety on the Si atom which bears the nitrogen-containing group. In formula (Si-III), R2 denotes a hydroxyl group or a C1-4 alkoxy group, whereas the residue in formula (Si-IV) is a methyl group. The individual Si moieties, which are labeled with the indices m and n or p and q, need not be present as blocks; instead, the individual units can also be distributed randomly, i.e., in the formulas (Si-III) and (Si-IV), each R1-Si(CH₃)₂ group is not necessarily bound to a —[O—Si(CH₃)₂] moiety.

Agents according to the invention which contain at least one amino-functional silicone polymer (a3) of the formula (Si-V) have also proven to be particularly effective with respect to the desired effects:

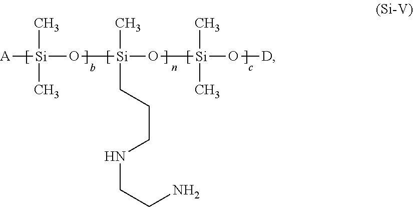

where
A represents an —OH, —O—Si(CH₃)₃, —O—Si(CH₃)₂OH, or —O—Si(CH₃)₂OCH₃ group,
D represents an —H, —Si(CH₃)₃, —Si(CH₃)₂OH, or —Si(CH₃)₂OCH₃ group,
b, n, and c stand for integers between 0 and 1000,
with the provisos that
   n>0 and b+c>0
   at least one of the conditions A=—OH or D=—H is met.

In the aforementioned formula (Si-V), the individual siloxane units having the indices b, c, and n are randomly distributed, i.e. they are not necessarily block copolymers.

The agent (a) can also comprise one or more different amino-functionalized silicone polymers which are described by the formula (Si-VI):

M(RₐQ_{b}SiO_{(4-a-b)/2)x}(R_{c}SiO_{(4-c)/2)y}M (Si-VI)
wherein, in the above formula, R is a hydrocarbon or a hydrocarbon group having 1 to approximately 6 carbon atoms, Q is a polar group of general formula —R¹HZ, in which R¹ is a bivalent linking group bonded to hydrogen and the group Z, composed of carbon and hydrogen atoms, carbon, hydrogen, and oxygen atoms, or carbon, hydrogen and nitrogen atoms, and Z is an organic, aminofunctional group containing at least one aminofunctional group; “a” assumes values in a range of from approximately 0 to approximately 2, “b” assumes values in a range of from approximately 1 to approximately 3, “a”+“b” is less than or equal to 3, and “c” is a number in a range of from approximately 1 to approximately 3, and x is a number in a range of from 1 to approximately 2,000, preferably approximately 3 to approximately 50, and most preferably approximately 3 to approximately 25, and y is a number in a range of from approximately 20 to approximately 10,000, preferably approximately 125 to approximately 10,000, and most preferably approximately 150 to approximately 1,000, and M is a suitable silicone end group as is known in the prior art, preferably trimethylsiloxy. Non-limiting examples of the groups represented by R include alkyl groups, such as methyl, ethyl, propyl, isopropyl, isopropyl, butyl, isobutyl, amyl, isoamyl, hexyl, isohexyl, and the like; alkenyl groups such as vinyl, halovinyl, alkylvinyl, allyl, haloallyl, and alkylallyl; cycloalkyl groups such as cyclobutyl, cyclopentyl, cyclohexyl, and the like; phenyl groups; benzyl groups; halohydrocarbon groups such as 3-chloropropyl, 4-bromobutyl, 3,3,3-trifluoropropyl, chlorocyclohexyl, bromophenyl, chlorophenyl, and the like; and sulfur-containing groups such as mercaptoethyl, mercaptopropyl, mercaptohexyl, mercaptophenyl, and the like; R is preferably an alkyl group containing 1 to approximately 6 carbon atoms, and most preferably R is methyl. Examples of R¹ include methylene, ethylene, propylene, hexamethylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂CH₂CH₂—, —CH₂CH(CH₃)C(O)OCH₂—, —(CH₂)₃C(O)OCH₂CH₂—, —C₆H₄C₆H₄—, —C₆H₄CH₂C₆H₄—, and —(CH₂)₃C(O)SCH₂CH₂—.

Z is an organic amino-functional group containing at least one functional amino group. A possible formula for Z is NH(CH₂)_{z}NH₂, where z is 1 or more. Another possible formula for Z is —NH(CH₂)_{z}(CH₂)_{zz}NH, in which both z and zz are independently 1 or more, wherein this structure comprises diamino ring structures, such as piperazinyl. Z is most preferably a —NHCH₂CH₂NH₂ group. Another possible formula for Z is N(CH₂)_{z}(CH₂)_{zz}NX₂ oder —NX₂, where each X of X₂ is selected independently from the group consisting of hydrogen and alkyl groups having 1 to 12 carbon atoms, and zz is 0.

Q is most preferably a polar, amino-functional group of formula —CH₂CH₂CH₂NHCH₂CH₂NH₂. In the formulas, “a” assumes values in the range of from approximately 0 to approximately 2, “b” assumes values in the range of from approximately 2 to approximately 3, “a”+“b” is less than or equal to 3, and “c” is a number in the range of from approximately 1 to approximately 3. The molar ratio of the RₐQ_{b}SiO_{(4-a-b)/2} units to the R_{c}SiO_{(4-c)/2} units lies within a range of from approximately 1:2 to 1:65, preferably approximately 1:5 to approximately 1:65, and most preferably approximately 1:15 to approximately 1:20. If one or more silicones of the above formula are used, then the various variable substituents in the above formula can be different in the various silicone components, present in the silicone mixture.

Within the scope of another preferred embodiment, an agent according to the invention is characterized in that it contains at least one amino-functional silicone polymer of the formula (Si-VII):

R′ₐG₃₋ₐ-Si(OSiG₂)ₙ-(OSiG_{b}R′_{2-b})ₘ—O—SiG₃₋ₐ-R′ₐ (Si-VII),

in which:
G is —H, a phenyl group, —OH, —O—CH₃, —CH₃, —O—CH₂CH₃, —CH₂CH₃, —O—CH₂CH₂CH₃, —CH₂CH₂CH₃, —O—CH(CH₃)₂, —CH(CH₃)₂, —O—CH₂CH₂CH₂CH₃, —CH₂CH₂CH₂CH₃, —O—CH₂CH(CH₃)₂, —CH₂CH(CH₃)₂, —O—CH(CH₃)CH₂CH₃, —CH(CH₃)CH₂CH₃, —O—C(CH₃)₃, or —C(CH₃)₃;
a represents a number between 0 and 3, in particular 0;
b represents a number between 0 and 1, in particular 1;
m and n are numbers whose sum (m+n) is between 1 and 2000, preferably between 50 and 150, n preferably assuming values of 0 to 1999 and in particular from 49 to 149, and m preferably assuming values of 1 to 2000, in particular from 1 to 10;
R′ is a monovalent group selected from:
   -Q-N(R″)—CH₂—CH₂—N(R″)₂
   -Q-N(R″)₂
   -Q-N⁺(R″)₃A⁻
   -Q-N⁺H(R″)₂A⁻
   -Q-N⁺H₂(R″)A⁻
   -Q-N(R″)—CH₂—CH₂—N⁺R″H₂A^{-,}
      where each Q represents a chemical bond, —CH₂—, —CH₂—CH₂—, —CH₂CH₂CH₂—, —C(CH₃)₂—,
      —CH₂CH₂CH₂CH₂—, —CH₂C(CH₃)₂—, or —CH(CH₃)CH₂CH₂—,
      R″ represents identical or different functional groups from the group —H, -phenyl, -benzyl, —CH₂—CH(CH₃)Ph, from the C₁₋₂₀ alkyl groups, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, and A represents an anion preferably selected from chloride, bromide, iodide, or methosulfate.

Within the scope of another preferred embodiment, an agent according to the invention is characterized in that it contains at least one amino-functional silicone polymer (a3) of the formula of the formula (Si-VIIa):

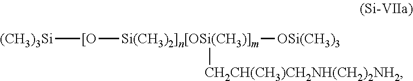

in which m and n are numbers of which the sum (m+n) is between 1 and 2000, preferably between 50 and 150, n preferably assuming values of 0 to 1999 and in particular from 49 to 149, and m preferably assuming values of 1 to 2000, in particular from 1 to 10.

These silicones are designated as trimethylsilylamodimethicones in accordance with the INCI Declaration.

In another preferred embodiment, an agent according to the invention is characterized in that it contains at least one amino-functional silicone polymer of the formula (Si-VIIb):

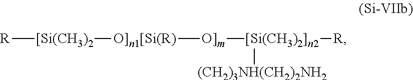

in which R denotes —OH, —O—CH₃, or a —CH₃ group, and m, n1, and n2 are numbers whose sum (m+n1+n2) amounts to between 1 and 2000, preferably between 50 and 150, the sum (n1+n2) preferably assuming values from 0 to 1999 and in particular from 49 to 149, and m preferably assuming values from 1 to 2000, in particular from 1 to 10.

According to the INCI Declaration, these amino-functionalized silicone polymers are referred to as amodimethicones.

Irrespective of which amino-functional silicones are used, agents according to the invention are preferred that contain an amino-functional silicone polymer (a3) of which the amine value is above 0.25 meq/g, preferably above 0.3 meq/g, and in particular above 0.4 meq/g. The amine value here represents the milliequivalents of amine per gram of the amino-functional silicone. Said value can be determined by titration and may also be given in the unit mg KOH/g.

Furthermore, agents which contained a specific 4-morpholinomethyl-substituted silicone polymer (a3) are also suitable. This amino-functionalized silicone polymer comprises structural units of formulas (Si-VIII) and of formula (Si-IX):

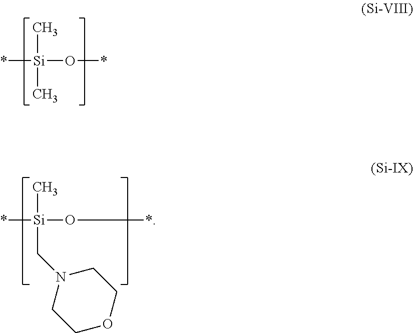

Corresponding 4-morpholinomethyl-substituted silicone polymers are described below.

A preferred amino-functionalized silicone polymer is known under the name of amodimethicone/morpholinomethyl silsesquioxane copolymer, and is commercially available in the form of the raw material Belsil ADM 8301 E by Wacker.

For example, a silicone which has structural units of formulas (Si-VIII), (Si-IX) and (Si-X) can be used as 4-morpholinomethyl-substituted silicone:

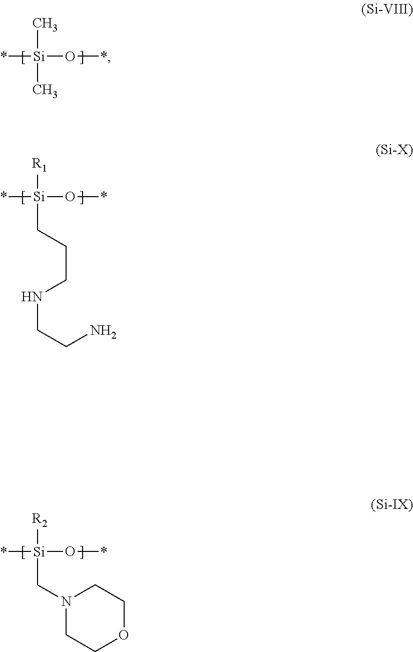

in which
R1 represents —CH₃, —OH, —OCH₃, —O—CH₂CH₃, —O—CH₂CH₂CH₃, or —O—CH(CH₃)₂;
R2 represents —CH₃, —OH, or —OCH₃.

Particularly preferred agents according to the invention contain at least one 4-morpholinomethyl-substituted silicone of formula (Si-XI):

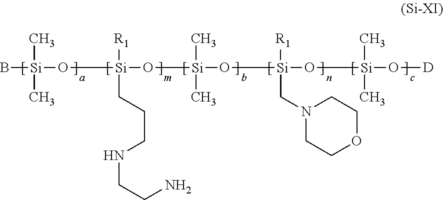

in which
R1 represents —CH₃, —OH, —OCH₃, —O—CH₂CH₃, —O—CH₂CH₂CH₃, or —O—CH(CH₃)₂;
R2 represents —CH₃, —OH, or —OCH₃.
B represents an —OH, —O—Si(CH₃)₃, —O—Si(CH₃)₂OH, or —O—Si(CH₃)₂OCH₃ group,
D represents an —H, —Si(CH₃)₃, —Si(CH₃)₂OH, or —Si(CH₃)₂OCH₃ group,
a, b, and c represent, independently of one another, integers between 0 and 1000, with the proviso that a+b+c>0
m and n represent, independently of one another, integers between 1 and 1000, with the proviso that
   at least one of the conditions B=—OH or D=—H is met,
   the units a, b, c, m, and n are distributed randomly or in blocks in the molecule.

Structural formula (Si-XI) is intended to indicate that the siloxane groups n and m do not necessarily have to be directly bonded to an end group B or D. Instead, in preferred formulas (Si-VI), a>0 or b>0 and, in particularly preferred formulas (Si-VI), a>0 and c>0; i.e., the terminal group B or D is preferably bonded to a dimethylsiloxy group. In formula (Si-VI) as well, the siloxane units a, b, c, m, and n are preferably distributed randomly.

The silicones represented by formula (Si-VI) and used according to the invention can be trimethylsilyl-terminated (D or B=—Si(CH₃)₃), but they may also be dimethylsilylhydroxy-terminated at both ends or dimethylsilylhydroxy- and dimethylsilylmethoxy-terminated at one end. Within the context of the present invention, silicones which are particularly preferably used are selected from silicones in which:
B=—O—Si(CH₃)₂OH and D=—Si(CH₃)₃
B=—O—Si(CH₃)₂OH and D=—Si(CH₃)₂OH
B=—O—Si(CH₃)₂OH and D=—Si(CH₃)₂OCH₃
B=—O—Si(CH₃)₃ and D=—Si(CH₃)₂OH
B=—O—Si(CH₃)₂OCH₃ and D=—Si(CH₃)₂OH.
   These silicones lead to enormous improvements in the hair properties of hair treated with the agents according to the invention, and to greatly improved protection during oxidative treatment.

It has been found to be particularly advantageous if the agent according to the invention contains the amino-functionalized silicone polymer(s) (a3) in certain quantity ranges. Particularly good results were obtained when the agent contained—based on the total weight of the agent—one or more amino-functionalized silicone polymers (a3) in a total amount of 0.1 to 8.0% by weight, preferably 0.2 to 5.0% by weight, more preferably 0.3 to 3.0% by weight, and very particularly preferably 0.4 to 2.5% by weight.

Within the scope of another particularly preferred embodiment, an agent according to the invention is characterized in that it contains—based on the total weight of the agent—one or more amino-functionalized silicone polymers (a3) in a total amount of 0.1 to 8.0% by weight, preferably 0.2 to 5.0% by weight, more preferably 0.3 to 3.0% by weight, and very particularly preferably 0.4 to 2.5% by weight.

### Average Water Content

The agent described above is a ready-to-use agent which can be applied to the keratinous material. This ready-to-use agent preferably possesses a low to average water content. It has been found that particularly those agents are well-suited which contain—based on the total weight of the agent—0.1 to 70.0% by weight, preferably 0.5 to 35.0% by weight, more preferably 1.0 to 20.0% by weight, and particularly preferably 1.5 to 7.5% by weight water.

In another explicitly very particularly preferred embodiment, an agent according to the present invention is characterized in that it contains—based on the total weight of the agent—0.1 to 70.0% by weight, preferably 0.5 to 35.0% by weight, more preferably 0.1 to 20.0% by weight, and especially preferably 1.5 to 7.5% by weight water.

### Average Solvent

The use of further solvents which are different from the compounds of group (a1) has led to good results. For this reason, the agent according to the invention can additionally contain at least one solvent as an optional component.

Suitable solvents that can be used are, for example, solvents from the group consisting of 1,2-propylene glycol, 1,3-propylene glycol, 1,2-butylene glycol, dipropylene glycol, ethanol, isopropanol, diethylene glycol monoethyl ether, glycerol, phenoxyethanol, and benzyl alcohol. The use of 1,2-propylene glycol is very particularly preferred.

In a further very particularly preferred embodiment, an agent according to the invention is characterized in that it contains at least one solvent from the group consisting of 1,2-propylene glycol, 1,3-propylene glycol, 1,2-butylene glycol, dipropylene glycol, ethanol, isopropanol, diethylene glycol monoethyl ether, glycerol, phenoxyethanol, and benzyl alcohol, and very particularly preferably 1,2-propylene glycol.

Alternatively, 1,2-propylene glycol is also referred to as 1,2-propanediol and bears the CAS numbers 57-55-6 [(RS)-1,2-dihydroxypropane], 4254-14-2 [(R)-1,2-dihydroxypropane], and 4254-153 [(S)-1,2-dihydroxypropane]. Ethylene glycol is alternatively also referred to as 1,2-ethanediol and bears the CAS number 107-21-1. Glycerol is alternatively also referred to as 1,2,3-propanetriol and bears the CAS number 56-81-5. Phenoxyethanol has the CAS number 122-99-6.

All the solvents described above are commercially available from various chemicals suppliers such as Aldrich or Fluka.

By using the abovementioned solvents in suitable use quantities, a particularly stable agent can be obtained with which color results on the keratinous material with an especially high intensity can be obtained.

In a further preferred embodiment, an agent according to the invention is characterized in that it contains—based on the total weight of the agent—one or more solvents from the group consisting of 1,2-propylene glycol, 1,3-propylene glycol, 1,2-butylene glycol, dipropylene glycol, ethanol, isopropanol, diethylene glycol monoethyl ether, glycerol, phenoxyethanol, and benzyl alcohol, very particularly preferably 1,2-propylene glycol in a total amount of 1.0 to 60.0% by weight, preferably 5.0 to 55.0% by weight, more preferably 10.0 to 45.0% by weight, and very particularly preferably 20.0 to 40.0% by weight.

In another particularly preferred embodiment, an agent according to the present invention is characterized in that it contains—based on the total weight of the agent—one or more solvents in a total amount of 1.0 to 60.0% by weight, preferably 5.0 to 55.0% by weight, more preferably 10.0 to 45.0% by weight, and very particularly preferably 20.0 to 40.0% by weight 1,2-propylenglycol.

### Alkyl(poly)glycosides

As a further optional component, the agents according to the invention can additionally also contain at least one alkyl(poly)glycoside.

Within the scope of another preferred embodiment, an agent according to the invention is therefore characterized in that it contains at least one alkyl(poly)glycoside.

Alkyl(poly)glycosides (APG) are non-ionic surfactants which are produced completely from renewable raw materials (sugar components, predominantly glucose, for example from corn starch and fatty alcohol, for example from coconut oil). Alkyl polyglycosides are obtainable by an acid-catalyzed reaction (Fischer reaction) of sugars, in particular glucose (or starch), or of butyl glycosides with fatty alcohols.

Complex mixtures of alkyl monoglucoside (alkyl-α-d- and -β-d-glucopyranoside as well as small amounts of -glucofuranoside), alkyl diglucosides (-isomaltosides, -maltosides etc.), and alkyl oligoglucosides (-maltotriosides, -tetraosides etc.) are formed. The average degree of polymerization of commercial products whose alkyl groups are in the C8-C16 range is 1.2-1.5.

An alkyl monoglycoside which is obtained by reacting a molecule of a sugar component with a molecule of fatty alcohol can alternatively also be referred to as an alkyl glycoside. If the proportion of sugar components is greater than one, these are present as alkyl polyglycosides within the meaning of the present invention. The term alkyl(poly)glycoside with parentheses therefore refers to a mixture of alkyl monoglycosides and the corresponding oligomeric or higher polymeric compounds, the alkyl polyglycosides.

Particularly well suitable for achieving the object according to the invention are the alkyl(poly)glycosides of the formula (APG-1):

(Z)_{x′}—O—(CH₂)ₙₙ—CH₃ (APG-1)

where
Z is a sugar component from the group consisting of glucose, fructose, galactose, arabinose, ribose, xylose, lyxose, allose, altrose, mannose, gulose, idose, talose, and sucrose,
x′ is a number from 1 to 10, preferably a number from 1 to 5, and
nn is a number from 7 to 29, preferably a number from 7 to 21, more preferably a number from 11 to 21, and very particularly preferably a number from 11 to 17.

The Z group is a sugar component which is from the group consisting of glucose, fructose, galactose, arabinose, ribose, xylose, lyxose, allose, altrose, mannose, gulose, idose, talose, and sucrose.

In the formula (APG-1), there is a number of sugar components whose number corresponds to the value x′. If the index number x assumes a value greater than 1, several sugar components are present in the formula (APG-1). In principle, various sugar components can hereby also be included in each formula (APG-1) (in the event of x′=2, Z can be, for example, glucose and fructose). Any mono- or oligosaccharides can therefore be used as the sugar component Z. Usually, sugars with 5 or 6 carbon atoms as well as the corresponding oligosaccharides are used. Such sugars are, for example, glucose, fructose, galactose, arabinose, ribose, xylose, lyxose, allose, altrose, mannose, gulose, idose, talose, and sucrose.

The index number x′ is a number from 1 to 10, preferably a number from 1 to 5.

The index number nn characterizes the length of the alkyl chain of the alkyl(poly)glycoside. nn stands for a number from 7 to 29, preferably a number from 7 to 21, more preferably a number from 11 to 21, and very particularly preferably a number from 11 to 17. The total length of the alkyl chain is nn+1.

Within the scope of an another embodiment, a particularly preferred agent is characterized in that it contains:
(a6) at least one alkyl(poly)glycoside of formula (APG-1),

(Z)_{x′}—O—(CH₂)ₙₙ—CH₃ (APG-1)
where
Z is a sugar component from the group consisting of glucose, fructose, galactose, arabinose, ribose, xylose, lyxose, allose, altrose, mannose, gulose, idose, talose, and sucrose,
x′ is a number from 1 to 10, preferably a number from 1 to 5, and
nn is a number from 7 to 29, preferably a number from 7 to 21, more preferably a number from 11 to 21, and very particularly preferably a number from 11 to 17.

According to the invention, alkyl polyglycosides corresponding to the general formula RO—(Z)_{x′} are used, wherein R denotes an alkyl, Z denotes a sugar, and x′ denotes the number of sugar units.

Those alkyl polyglycosides are particularly preferred in which R:
substantially consists of C₈- and C₁₀-alkyl groups (i.e. in the formula (APG-1), nn is an integer from 7 to 9), or
substantially consists of C₁₂- and C₁₄-alkyl groups (i.e. in the formula (APG-1), nn is an integer from 11 to 13), or
substantially consists of C₈- to C₁₈-alkyl groups, i.e. in the formula (APG-1), nn is an integer from 7 to 17), or
substantially consists of C₁₂- to C₁₆-alkyl groups (i.e. in the formula (APG-1), nn is an integer from 11 to 15), or
substantially consists of C₁₆ to C₁₈-alkyl groups, i.e. in the formula (APG-1), nn is an integer from 15 to 17).

Preferred sugar components are glucose, fructose, galactose, arabinose, and sucrose. It is very particularly preferred if Z is glucose. In this event, the agent according to the invention contains an alkyl(poly)glucoside.

The alkyl(poly)glucosides (a1) of the general formula (APG-2) very particularly preferably contain:

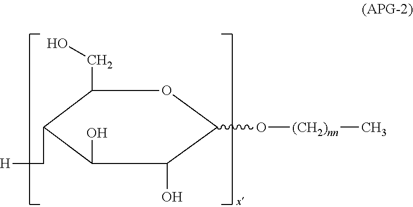

where
x′ is a number from 1 to 10, preferably a number from 1 to 5, and
nn is a number from 7 to 29, preferably a number from 7 to 21, more preferably a number from 11 to 21, and very particularly preferably a number from 11 to 17.

In the formula (APG-2), the index number x′ is a number from 1 to 10, preferably a number from 1 to 5.

The index number nn is a number from 7 to 29, preferably a number from 7 to 21, more preferably a number from 11 to 21, and very particularly preferably a number from 11 to 17.

In a further embodiment, a particularly preferred agent according to the invention is therefore characterized in that it contains:
(a6) at least one alkyl(poly)glucoside of formula (APG-2),

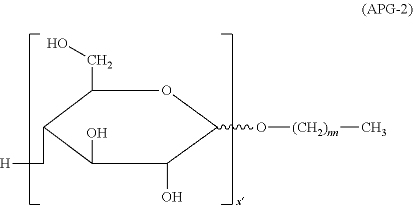

where
x′ is a number from 1 to 10, preferably a number from 1 to 5, and
nn is a number from 7 to 29, preferably a number from 7 to 21, more preferably a number from 11 to 21, and very particularly preferably a number from 11 to 17.

The alkylpolyglycosides which can be used according to the invention contain on average 1.1 to 5 sugar units. Alkylpolyglycosides having x′ values of 1.1 to 2.0 are preferred. Alkylglycosides in which x is on average 1.1 to 1.8 are very particularly preferred.

Very particularly preferred alkyl polyglucosides are those whose alkyl group is a lauryl group. With substance mixtures from natural sources, those sources are preferred which have a high proportion of C12 fatty acids, in particular coconut fatty acids. Accordingly, particularly preferred agents according to the invention are characterized in that they contain at least one alkyl(poly)glucoside of the formula (APG-2):

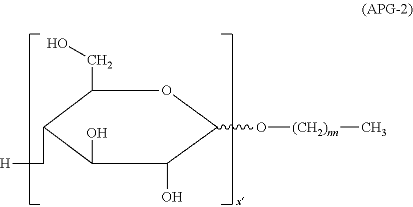

where
x′ is a number from 1.1 to 1.8, very particularly preferably from 1.2 to 1.5, and
nn is the number 11.

A very particularly suitable alkyl(poly)glucoside of this type is, for example, lauryl glucoside which bears the CAS number 110615-47-9 and can be purchased commercially from BASF under the trade names Plantacare 1200 UP, Plantaren 1200 UP, and Plantaren 1200 N.

The alkyl(poly)glycosides are preferably used within certain quantity ranges. It has proven to be particularly advantageous if the agent contains—based on the total weight of the agent—one or more alkyl (poly)glycosides (a1) in a total amount of 0.01 to 20.0% by weight, preferably 0.05 to 15.0% by weight, more preferably 0.1 to 10.0% by weight, still more preferably 0.15 to 8.0% by weight, and very particularly preferably 0.2 to 6.0% by weight.

In another particularly preferred embodiment, an agent according to the invention is characterized in that it contains—based on the total weight of the agent—one or more alkyl(poly)glycosides in a total amount of 0.01 to 20.0% by weight, preferably 0.05 to 15.0% by weight, more preferably 0.1 to 10.0% by weight, still more preferably 0.15 to 8.0% by weight, and very particularly preferably 0.2 to 6.0% by weight.

### Further Optional Components in the Agent

Depending on the desired form of production, the agent according to the invention can optionally also contain additional components or ingredients.

### Additional Ingredients in the Agent

The agents may also contain still more active ingredients, auxiliaries, and additives, such as structurants such as fatty components, glucose, maleic acid, and lactic acid; hair-conditioning compounds such as phospholipids, for example lecithin and kephalins; perfume oils; dimethyl isosorbide and cyclodextrins; polymers such as anionic, nonionic, and cationic polymers; surfactants such as anionic, nonionic, cationic, zwitterionic, and amphoteric surfactants; fatty components; fiber structure-improving active ingredients, in particular mono-, di-, and oligosaccharides such as glucose, galactose, fructose, fruit sugar, and lactose; dyes for coloring the agent; anti-dandruff active ingredients such as piroctone olamine, zinc omadine, and climbazole; amino acids and oligopeptides; protein hydrolysates based on animals and/or vegetables, as well as in the form of their fatty acid condensation products or optionally anionically or cationically modified derivatives; vegetable oils; light stabilizers and UV blockers; active ingredients such as panthenol, pantothenic acid, pantolactone, allantoin, pyrrolidinonecarboxylic acids and their salts, and bisabolol; polyphenols, in particular hydroxycinnamic acids, 6,7-dihydroxycoumarins, hydroxybenzoic acids, catechins, tannins, leucoanthocyanidins, anthocyanidins, flavanones, flavones, and flavonols; ceramides or pseudoceramides; vitamins, provitamins, and vitamin precursors; plant extracts; fats and waxes such as fatty alcohols, beeswax, montan wax, and paraffins; swelling and penetrating agents such as glycerol, propylene glycol monoethyl ether, carbonates, hydrogen carbonates, guanidines, ureas, and primary, secondary, and tertiary phosphates; opacifiers such as latex, styrene/PVP, and styrene/acrylamide copolymers; pearlescent agents such as ethylene glycol mono- and distearate and PEG-3 distearate; and propellants such as propane-butane mixtures, N₂O, dimethyl ether, CO₂, and air.

The selection of these additional substances is made by the person skilled in the art according to the desired properties of the agents. With respect to other optional components and the employed amounts of said components, reference is made expressly to relevant manuals known to the person skilled in the art. The additional active ingredients and auxiliaries are used in the preparations according the invention, preferably in amounts of, in each instance, 0.0001 to 25% by weight, in particular of 0.0005 to 15% by weight, relative to the total weight of the particular agent.

### Methods for Dyeing Keratinous Materials

The agents described above can be used outstandingly in methods for dyeing keratinous material, in particular human hair.

A second subject matter of the present invention is therefore a method for dyeing keratinous material, in particular human hair, in which an agent as disclosed in detail in the description of the first subject matter of the invention is applied to the keratinous fibers and, if necessary, rinsed out again after an exposure time of 30 seconds to 45 minutes.

A second subject matter of the invention is therefore a method for dyeing keratinous material, in particular human hair, comprising the following steps:
(1) applying a coloring agent on the keratinous material, wherein the coloring agent is an agent as has been disclosed in detail in the description of the first subject matter of the invention,
(2) exposing the coloring agent on the keratinous material, and
(3) rinsing out the coloring agent with water.

In step (1) of the method according to the invention, the agent of the first subject matter of the invention is applied to the keratinous material, which is very particularly preferably human hair.

In step (2) of the method according to the invention, the agent is then allowed to act on the keratinous material after its application. In this context, various exposure times of, for example, 30 seconds to 60 minutes are conceivable.

However, a great advantage of the dyeing system according to the invention is that an intensive color result can be achieved even in very short periods after short exposure times. For this reason, it is advantageous for the application mixture to remain on the keratinous material after application only for comparatively short periods of 30 seconds to 15 minutes, preferably 30 seconds to 10 minutes, and particularly preferably 1 to 5 minutes.

In another preferred embodiment, a method according to the invention is characterized by
(2) exposing the keratinous material to the coloring agent for a period of 30 seconds to 15 minutes, preferably 30 seconds to 10 minutes, and more preferably 1 to 5 minutes.

After the action of the application mixture on the keratinous material, said keratinous material is rinsed with water in step (3) of the process.

In one embodiment, the application mixture can be washed out with water only, i.e. without the aid of an after-treatment agent or a shampoo. The use of an after-treatment agent or conditioner in step (6) is also conceivable in principle.

To achieve the object according to the invention, and to increase the application comfort, however, it has been found to be very particularly preferred to rinse out the agent in step (3) exclusively with water, without the aid of an additional aftertreatment agent, shampoos, or conditioners.

In another preferred embodiment, a method according to the invention is characterized by
(3) rinsing out the coloring agent exclusively with water.
   Method for dyeing keratinous material in which the ready-to-use agent is first produced.

As previously described, the agent of the first subject matter of the invention is a ready-to-use agent which is either provided directly to the user in its ready-to-use form, or which is prepared by mixing various agents just prior to use.

In order to ensure a particularly fine distribution of the pigments, it has been found to be very particularly preferred to produce the ready-to-use agent shortly before application, by mixing two or three different agents.

Within the scope of a particularly preferred embodiment, the ready-to-use agent is accordingly prepared by mixing at least two different agents, wherein the first of these two agents comprises the mixture of at least alkylene glycol of formula (AG) (a1) and pigment(s) (a2). For example, the mixture of alkylene glycol of formula (AG) (a1) and pigment(s) (a2) can represent a predispersion. The second agent contains at least one amino-functionalized silicone polymer (a3). To produce the ready-to-use agent, the two aforementioned agents are then shaken or stirred with one another.

Another subject matter of the present application is therefore a method for dyeing keratinous material, in particular human hair, comprising the following steps:
(1) providing an agent (I), wherein the agent (I) contains:
   (a1) at least one alkylene glycol of formula (AG) and
   (a2) at least pigment,
(2) providing an agent (II), wherein the agent (II) contains:
   (a3) at least one amino-functionalized silicone polymer.
(3) producing an application mixture by mixing the agents (I) and (II),
(4) applying the application mixture produced in step (3) on the keratinous material,
(5) exposing the keratinous material to the application mixture applied in step (4), and
(6) rinsing out the application mixture with water,
   wherein the ingredients (a1), (a2) and (a3) have already been disclosed in detail in the description of the first subject matter of the invention.

The agent (I) is preferably a predispersion of the pigments (a2) in the alkylene glycol(s) of formula (AG) (a1) which can, for example, be in the form of a concentrate.

If the agents (I) and (II) are mixed, the predispersion of the pigments (a2) in the alkylene glycol of formula (AG) (a1) ensures a particularly fine distribution of the pigments which is maintained even after the two agents (I) and (II) are mixed in the ready-to-use agent.

Finally, in the context of an additional embodiment, it can also prove advantageous to separate the components (a1) and (a2) from one another, so that the three components (a1), (a2) and (a3) which are previously separated from one another are mixed with each other during the preparation of the ready-to-use agent. This form of production can be advantageous, for example, when the pigment(s) (a2) are to be used in smaller amounts and/or in the form of a powder. The preparation of the pigments in powder form simplifies the quantitative transfer of the pigments into a mixing vessel or another container.

Another subject matter of the present application is therefore a method for dyeing keratinous material, in particular human hair, comprising the following steps:
(1) providing an agent (I), wherein the agent (I) contains:
   (a1) at least one alkylene glycol of formula (AG),
(2) providing an agent (II), wherein the agent (II) contains:
   (a2) at least pigment,
(3) providing an agent (III), wherein the agent (III) contains:
   (a3) at least one amino-functionalized silicone polymer,
(4) preparing an application mixture by mixing agents (I) and (II) and (III),
(5) applying the application mixture produced in step (4) on the keratinous material,
(6) exposing the keratinous material to the application mixture applied in step (5), and
(7) rinsing out the application mixture with water.
   wherein the ingredients (a1), (a2) and (a3) have already been disclosed in detail in the description of the first subject matter of the invention.

In this case, agent (I) contains at least one alkylene glycol of formula (AG) (a1) and can be present, for example, in the form of a concentrate.

The agent (II) contains at least one pigment (a2). In one possible type of production, the pigment is provided, for example, in the form of a powder.

The agent (III) contains at least one amino-functionalized silicone polymer (a3) and is preferably present in the form of a concentrate.

### Multi-Component Packaging Unit

To increase user comfort, the above-described agents in the form of a multicomponent packaging unit can be provided to the user.

Another subject matter is therefore a multicomponent packaging unit (kit of parts) for dyeing keratinous material, in particular human hair, comprising separately prepared:
a first container with an agent (I), wherein the agent (I) contains:
(a1) at least one alkylene glycol of formula (AG) and
(a2) at least pigment,
a second container with an agent (II), wherein the agent (II) contains:
(a3) at least one amino-functionalized silicone polymer,
   wherein the ingredients (a1), (a2) and (a3) have already been disclosed in detail in the description of the first subject matter of the invention.

Another subject matter of the present invention is therefore a multicomponent packaging unit (kit of parts) for dyeing keratinous material, in particular human hair, comprising separately prepared:
a first container with an agent (I), wherein the agent (I) contains:
(a1) at least one alkylene glycol of formula (AG),
a second container with an agent (II), wherein the agent (II) contains:
(a2) at least pigment,
a third container with an agent (III); wherein the agent (III) contains:
(a3) at least one amino-functionalized silicone polymer,
   wherein the ingredients (a1), (a2) and (a3) have already been disclosed in detail in the description of the first subject matter of the invention.

Concerning the additional preferred embodiments of the methods according to the invention and the multicomponent packaging unit according to the invention, what has been said about the agent according to the invention applies mutatis mutantis.

### EXAMPLES

### 1. Formulations

The following ready-to-use coloring agents were prepared (all data are in percent by weight unless stated otherwise):

| Ready-to-use coloring agent | E1 |
|---|---|
| Plantacare 2000 UP (Decyl Glucoside, | 0.38 |
| CAS number 110615-47-9, BASF) | |
| Water (distilled) | 3.02 |
| Unipure Red LC 3071 (CI 15850, | 0.38 |
| Sensient) (a2) | |
| Aminosilicone (Dow Corning 2-8566 | 0.74 |
| (Siloxanes and Silicones, 3-[(2- | |
| Aminoethyl)amino]-2-methylpropyl | |
| Me, Di-Me-Siloxane) (a3) | |
| PEG 6000 (a12) (polyethylene glycol, | 9.93 |
| molecular weight 6000 to 7500 g/mol) | |
| PEG 400 (a11) (polyethylene glycol, | to 100 |
| molecular weight 400 g/mol) | |

### 2. Application

The ready-to-use agents E1 and E2 produced beforehand were applied to hair strands (Kerling, type “Euronatur hair white” (ENH)) (bath ratio: 1 g agent per g of hair strand) and allowed to act for three minutes. Subsequently, the hair strands were washed thoroughly (1 minute) with water and dried.

### 3. Measurement of Wash Fastness

After drying, the colored strands were measured using a colorimeter from Datacolor, type Spectraflash 450.

Each strand was then washed three times manually. To this end, each strand was moistened with water, then a commercial shampoo (Schauma 7-Kräuter) was applied to the strand (0.25 g shampoo per 1 g of hair) and massaged with the fingers 30 seconds. The strand was then rinsed for 1 minute under running lukewarm water, and the strand of hair was dried. The process described above corresponds to hair washing. For each additional hair wash, the process was repeated. The strands were colorimetrically measured again after 3 and after 6 hair washes.

The dE value used for the assessment of wash fastness is derived from the L*a*b* colorimetric values measured from the respective strand as follows:

dE=[(Lᵢ−L₀)²+(aᵢ−a₀)²+(bᵢ−b₀)]^{1/2}
L₀, a₀ and b₀=measured values directly after dyeing
Lᵢ, aᵢ and bᵢ=measured values after 3 hair washes (or after 6 hair washes)

The smaller the dE value, the less the color difference compared to the dyed, unwashed hair, and the better the wash fastness.

FA describes the percent color content after the corresponding number of hair washes and is calculated according to the following formula:

$$F A [ \% ] = 100 * ( \frac{Δ E ( u - g )}{Δ E ( u - c )} )$$

FA=color retention in [%]
u=untreated starting hair
g=washed colored hair
c=colored hair

The higher the color content, the better the wash fastness.

| | E1 |
|---|---|
| dE (uncolored versus colored) | 60.40 |
| dE (colored, after 3 hair washes) | 26.10 |
| FA after 3 hair washes [%] | 64.00 |
| dE (colored, after 6 hair washes) | 35.40 |
| FA after 6 hair washes [%] | 47.00 |

With the agent E1, the hair strands were able to be intensively colored and had a very good wash fastness and a high color content.

1. An agent for dyeing keratinous material, wherein the agent comprises:
at least one alkylene glycol of formula (AG) 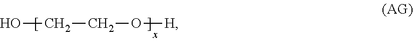
where x is an integer ranging from 1 to 10,000;
at least one pigment; and
at least one amino-functionalized silicone polymer.

2. The agent according to claim 1,
wherein x is an integer ranging from 1 to 100.

3. The agent according to claim 1,
wherein x is an integer ranging from 101 to 1000.

4. The agent according to claim 1, wherein the at least one alkylene glycol—based on the total weight of the agent—is present in an amount ranging from 10.0 to 99.0% by weight.

5. The agent according to claim 1, wherein:
the at least one alkylene glycol—based on the total weight of the agent—is present in an amount ranging from 20.0 to 99.0% by weight; and/or
at least one or more alkylene glycols of formula (AG), where x is an integer ranging from 1 to 100, in a total amount ranging from 1.0 to 35.0% by weight based on the total weight of the agent.

6. The agent according to claim 1, wherein the at least one pigment is selected from the group comprising colored metal oxides, metal hydroxides, metal oxide hydrates, silicates, metal sulfides, complex metal cyanides, metal sulfates, bronze pigments, and/or from mica-based colored pigments which are coated with at least one metal oxide and/or a metal oxychloride, or combinations thereof.

7. The agent according to claim 1, wherein the at least one organic pigment is selected from the group comprising:
carmine;
quinacridone;
phthalocyanine;
sorghum;
blue pigments with the Color Index numbers CI 42090, CI 69800, CI 69825, CI 73000, CI 74100, CI 74160, or combinations thereof;
yellow pigments with the Color Index numbers CI 11680, CI 11710, CI 15985, CI 19140, CI 20040, CI 21100, CI 21108, CI 47000, CI 47005, or combinations thereof;
green pigments with the Color Index numbers CI 61565, CI 61570, CI 74260, or combinations thereof;
orange pigments with the Color Index numbers CI 11725, CI 15510, CI 45370, CI 71105, or combinations thereof;
red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915, CI 75470, or combinations thereof; or
combinations thereof.

8. The agent according to claim 1, wherein the at least one pigment—based on the total weight of the composition—is present in an amount ranging from 0.01 to 10.0% by weight.

9. The agent according to claim 1,
wherein the at least one amino-functionalized silicone polymer comprises at least one secondary amino group.

10. The agent according to claim 1, wherein the at least one amino-functionalized silicone polymer comprises at least one structural unit of the formula (Si-amino): 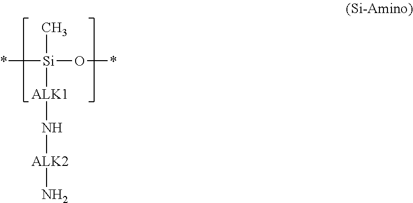
where
ALK1 and ALK2 represent, independently of one another, a linear or branched divalent C₁-C₂₀ alkylene group.

11. The agent according to claim 1, wherein the at least one amino-functionalized silicone polymer comprises structural units of the formula (Si-I) and of the formula (Si-II)

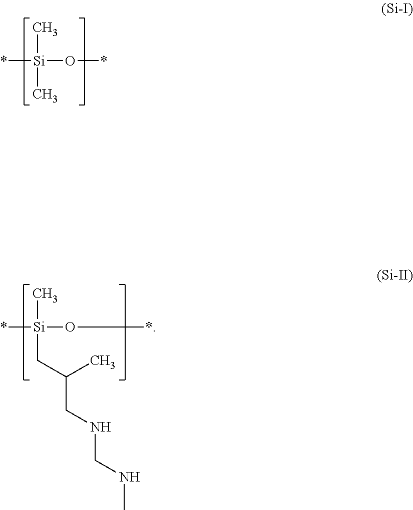

12. The agent according to claim 1, wherein the at least one amino-functionalized silicone polymer—based on the total weight of the composition—is present in an amount ranging from 0.1 to 8.0% by weight.

13. The agent according to claim 1, further comprising water—based on the total weight of the composition—in an amount ranging from 0.1 to 70.0% by weight.

14. The agent according to claim 1, further comprising at least one alkyl(poly)glycoside.

15. A method for dyeing keratinous material, wherein the method comprises:
applying the agent of claim 1 to the keratin material for a period of time ranging from 30 seconds to 45 minutes; and
optionally rinsing out the agent after the period of time.
